(19) United States
(12) Plant Patent Application Publication
(10) Pub. No.: US 2013/0247262 P1
(43) Pub. Date: Sep. 19, 2013

(54) Pelargonium plant named 'KLEPP11273'

(50) ;

(71) Applicants: Nils Klemm, Stuttgart (DE); Martin Glawe, Stuttgart (DE)

(72) Inventors: Nils Klemm, Stuttgart (DE); Martin Glawe, Stuttgart (DE)

(73) Assignee: KLEMM + SOHN GMBH & CO. KG, Stuttgart (DE)

(21) Appl. No.: 13/385,890

(22) Filed: Mar. 13, 2012

(51) Int. Cl. A01H 5/00

(52) U.S. Cl. PLT/327

(57) ABSTRACT

A new variety of pelargonium named ‘KLEPP11273’, particularly characterized by rich, early flowering, very good branching, medium growth, and salmon pink flowers, is disclosed.

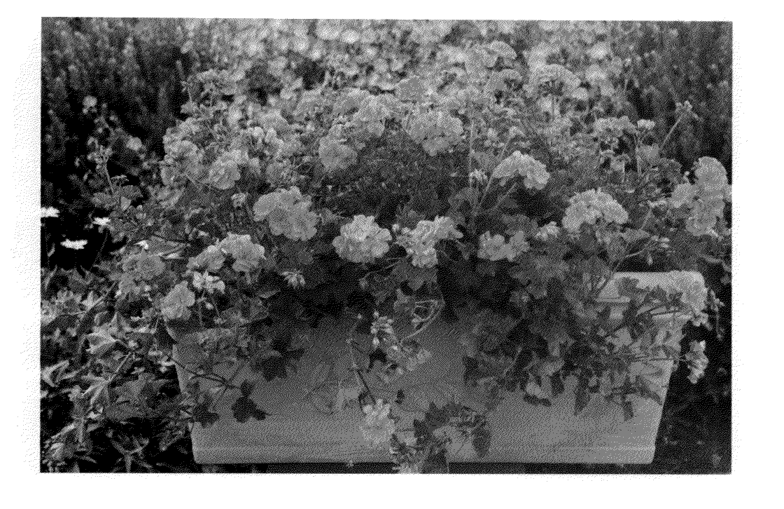

### GENUS AND SPECIES

Pelargonium peltatum

### VARIETY DENOMINATION

‘KLEPP11273’

### BACKGROUND OF THE NEW PLANT

The present invention comprises a new and distinct variety of Pelargonium, botanically known as Pelargonium peltatum, and hereinafter referred to by the variety name ‘KLEPP11273’. The new variety is the result of a controlled cross conducted in July 2005 in Stuttgart, Germany between the proprietary female Pelargonium parent, ‘KLEP02060’ (U.S. Plant Pat. No. 15,319), and the proprietary male Pelargonium parent, ‘KLEPP06122’ (U.S. Plant Pat. No. 19,502). A single plant selection was subsequently chosen for further evaluation and asexual propagation.

The new variety was first propagated via vegetative cuttings in August 2006 in Stuttgart, Germany and has been asexually reproduced repeatedly by vegetative cuttings in Stuttgart, Germany for approximately 6 to 8 generations. ‘KLEPP11273’ has been found to retain its distinctive characteristics through successive asexual propagations via vegetative cuttings.

Plant Breeder's Rights for this variety were applied for in the following countries: Canada on Mar. 4, 2011, Switzerland on Jul. 4, 2011, the European Union on Jul. 8, 2011, and Norway on Aug. 5, 2011. ‘KLEPP11273’ has not been made publicly available or sold more than one year prior to the filing date of this application.

### SUMMARY OF THE INVENTION

The following are the most outstanding and distinguishing characteristics of this new variety when grown under normal horticultural practices in a glass greenhouse in Stuttgart, Germany.

1. Very good branching;

2. Medium growth and early flowering time; and

3. Very rich flowering variety with salmon pink colored flowers.

### DESCRIPTION OF THE PHOTOGRAPH

This new Pelargonium plant is illustrated by the accompanying photograph which shows blooms, buds, and foliage of the plant. The colors shown are as true as can be reasonably obtained by conventional photographic procedures. The photograph is of a 3-month old plant taken in early Spring 2011 grown from a rooted cutting in a 12 centimeter pot in a glass greenhouse in Stuttgart, Germany under conditions which approximate those generally used in normal horticultural practice.

### DESCRIPTION OF THE NEW VARIETY

The following detailed descriptions set forth the distinctive characteristics of ‘KLEPP11273’. The data which define these characteristics were collected from asexual reproductions carried out in Stuttgart, Germany. The plant history was taken on 3-month old, un-pinched plants grown in 12 centimeter pots in a glass greenhouse in Stuttgart, Germany. The color readings were determined under natural light. Color references are to the RHS Colour Chart of The Royal Horticultural Society of London (RHS), Fifth Edition (2007).

### DETAILED BOTANICAL DESCRIPTION OF THE NEW PLANT

Classification:
   Family.—Geraniaceae.
   Species.—Pelargonium peltatum.
   Common name.—Pelargonium (Geranium).
   Denomination.—‘KLEPP11273’.
Parentage:
   Female parent.—Pelargonium plant named ‘KLEP02060’ (U.S. Plant Pat. No. 15,319).
   Male parent.—Pelargonium plant named ‘KLEPP06122’ (U.S. Plant Pat. No. 19,502).
Plant:
   Habit.—After planting upright, then hanging.
   Height to the top of the umbels.—25.0 cm to 30.0 cm.
   Spread.—Approximately 28.0 cm to 35.0 cm.
   Life cycle.—Spring to autumn.
   Form.—Round, bushy.
   Time to produce a rooted cutting.—16 to 18 days.
   Time to bloom from propagation.—Approximately 84 days.
   Lastingness of inflorescence on the plant.—10 days.
Stems:
   Stem color.—RHS 137D.
   Pubescence.—Present, short.
   Stem form.—Upright after planting, then hanging.
   Diameter.—0.3 cm to 0.8 cm.
   Length.—5.0 cm to 12.0 cm.
   Internode length.—0.5 cm to 1.3 cm.
Leaves:
   Arrangement.—Alternate.
   Shape.—Ivy-shaped with distinct lobes.
   Apex.—Round.
   Base.—Closed.
   Margin.—Crenated.
   Surface texture (both surfaces).—Matte.
   Length.—3.5 cm to 5.0 cm.
   Width.—4.0 cm to 6.0 cm.
   Color.—Upper surface: RHS 137A Lower surface: RHS 137B.
   Fragrance.—Absent.
   Surface pubescence.—Absent.
   Petiole.—Length: 13.0 cm Diameter: 0.3 cm Color: RHS 137D.
   Venation pattern.—Palmate.
   Venation color (both surfaces).—RHS 137D.
   Zonation.—Distance of center of zone to margin: 1.5 to 2.0 cm Color: RHS 187A.
Inflorescence:
   Flowering habit.—Continuously.
   Umbels.—Length: 10.0 cm. Diameter: 12.0 cm.
   Individual florets of the umbel.—Depth: 2.0 cm Diameter: 3.0 cm.
   Flowers per umbel.—2 to 3.
   Umbels per plant.—3 to 4.
   Fragrance.—Absent.
Flower buds:
   Cluster shape.—Round.
   Cluster size.—Length: 0.6 cm to 0.8 cm Width: 0.5 cm to 0.7 cm Buds per cluster: 7 to 12.
   Bud.—Length: 0.4 cm to 0.8 cm Diameter: 0.3 cm to 0.6 cm Shape: Ovate to elliptic Apex: Acute Base: Obtuse Margin: Entire Color: RHS 137C.
   Corolla.—Diameter: 4.0 cm Depth: 1.5 cm.
Petals:
   Quantity per flower.—7 to 11; petaloids present.
   Shape.—Spatulate.
   Apex.—Round, entire.
   Base.—Obtuse.
   Margin.—Entire.
   Length.—2.5 cm.
   Width.—2.0 cm.
   Texture (both surfaces).—Smooth.
Sepals:
   Quantity per flower.—5.
   Shape.—Elliptic.
   Apex.—Acute.
   Base.—Open.
   Margin.—Entire.
   Color.—Apex: RHS 138C Base: RHS 138D.
   Length.—1.5 cm to 2.0 cm.
   Width.—0.3 cm to 0.5 cm.
Pedicel:
   Length.—2.5 cm.
   Diameter.—0.2 cm.
   Color.—RHS 137C.
   Texture.—Smooth.
Peduncle:
   Length.—8.0 cm to 10.0 cm.
   Diameter.—0.3 cm.
   Color.—RHS 137C.
   Texture.—Smooth.
Reproductive organs:
   Stamens.—Quantity per flower: Moderate Anther length: 0.4 cm Filament length: 0.5 cm Filament color: RHS 155D Pollen amount: Moderate Pollen color: RHS 41D.
   Pistils.—Quantity per flower: 1 Stigma color: RHS 73D Style length: 0.4 cm Style color: RHS 155D Ovary arrangement: Upright Ovary color: RHS 140D.
Fruit and seed set: None observed.
Disease, pest, and stress resistance: No particular resistance observed.
Comparision with parental varieties: ‘KLEPP11273’ differs from the female parent, ‘KLEP02060’ (U.S. Plant Pat. No. 15,319), in that ‘KLEPP11273’ has salmon pink colored flowers and medium growth, while ‘KLEP02060’ has light pink flowers and stronger growth. ‘KLEPP11273’ differs from the male parent, ‘KLEPP06122’ (U.S. Plant Pat. No. 19,502), in that ‘KLEPP11273’ is a rich flowering plant and has very good branching, while ‘KLEPP06122’ has less rich flowering and medium branching.

We claim:

1. A new and distinct variety of pelargonium plant named ‘KLEPP11273’ as shown and described herein.
